(19) United States
(12) Patent Application Publication
(10) Pub. No.: US 2025/0134358 A1
(43) Pub. Date: May 1, 2025

(54) DEVICES, SYSTEMS, AND METHODS FOR APPLYING AN ELONGATE MEMBER TO A MEDICAL SCOPE

(71) Applicant: BOSTON SCIENTIFIC SCIMED, INC., Maple Grove, MN (US)

(72) Inventors: Jennifer Lewitzky, Lexington, MA (US); Colby Harris, Norfolk, MA (US); Austin Grant Johnson, Worcester, MA (US)

(73) Assignee: BOSTON SCIENTIFIC SCIMED, INC., Maple Grove, MN (US)

(21) Appl. No.: 18/925,934

(22) Filed: Oct. 24, 2024

Related U.S. Application Data
(60) Provisional application No. 63/593,114, filed on Oct. 25, 2023.

(51) Int. Cl. A61B 1/00

(52) U.S. Cl. CPC A61B 1/0014

(57) ABSTRACT

Devices, systems, and methods for applying an elongate member to a shaft of an endoscope. The system may include a flexible elongate member and a tool for applying the elongate member to the shaft of the endoscope. The tool may include an elongate support and a plurality of connectors. The plurality of connectors may be longitudinally spaced apart along the elongate support(s). The plurality of connectors may be releasably coupled with the flexible elongate member at two or more locations along the flexible elongate member when the flexible elongate member is in a helical configuration.

### CROSS-REFERENCE TO RELATED APPLICATIONS

This application claims the benefit of priority under 35 U.S.C. § 119 to U.S. Provisional Application No. 63/593,114, filed Oct. 25, 2023, the entire disclosure of which is hereby incorporated by reference herein for all purposes.

### TECHNICAL FIELD

This disclosure relates generally to assemblies and methods for applying elongate members to scopes, and particularly to systems for applying elongate members to a medical scope such as an endoscope.

### BACKGROUND

A wide variety of intracorporeal medical devices and systems have been developed for medical use, for example, for transluminal procedures. Some of these devices and systems include guidewires, catheters, catheter systems, transluminal instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a system for applying a flexible elongate member to a shaft of an endoscope may comprise an elongate support, a plurality of connectors longitudinally spaced apart along the elongate support, and a flexible elongate member, wherein the plurality of connectors may be releasably coupled with the flexible elongate member at two or more locations along the flexible elongate member when the flexible elongate member is in a helical configuration.

Alternatively or additionally to any of the examples above, the plurality of connectors may be releasably coupled with the flexible elongate member at a same circumferential location, longitudinally spaced apart along the flexible elongate member in the helical configuration.

Alternatively or additionally to any of the examples above, the system may further include a stop secured to an end of the flexible elongate member, and the stop may be configured to couple with an end of the shaft of the endoscope about which the flexible elongate member is applied and maintain a position of the end of the flexible elongate member relative to the shaft as the elongate support is moved longitudinally along the shaft and away from the end of the shaft.

Alternatively or additionally to any of the examples above, the stop may be one of an endoscope clip and a cap.

Alternatively or additionally to any of the examples above, the system may further include a plurality of longitudinally extending rows of the plurality of connectors, the plurality of longitudinally extending rows may be circumferentially spaced apart from one another.

Alternatively or additionally to any of the examples above, the plurality of longitudinally extending rows of connectors may comprise three longitudinally extending rows of connectors with each row circumferentially spaced equally apart about the flexible elongate member in the helical configuration.

Alternatively or additionally to any of the examples above, the plurality of connectors may be arranged in a single longitudinally extending row along the elongate support.

Alternatively or additionally to any of the examples above, the elongate support may be an elongate sheet of material defining the plurality of connectors.

Alternatively or additionally to any of the examples above, the system may further comprise a handle extending along the elongate support.

Alternatively or additionally to any of the examples above, one or more connectors of the plurality of connectors may be c-clips.

In a further example, a device may further comprise an elongate support and a plurality of connectors, wherein two or more of the plurality of connectors are longitudinally spaced in a row along the elongate support, wherein the plurality of connectors may be configured to be releasably coupled with a flexible elongate member at two or more locations along the flexible elongate member when the flexible elongate member is in a helical configuration.

Alternatively or additionally to any of the examples above, the elongate support may be a first elongate support and the row along the elongate support is a first row of the plurality of connectors extending along the elongate support and the device may further comprise two or more of the plurality of connectors in a second row of the plurality of connectors extending along a second elongate support and two or more of the plurality of connectors in a third row of the plurality of connectors extending along a third elongate support, wherein the first elongate support, the second elongate support, and the third elongate support may be circumferentially spaced one hundred twenty degrees apart from one another.

Alternatively or additionally to any of the examples above, the elongate support may be an elongate sheet of material defining the plurality of connectors.

Alternatively or additionally to any of the examples above, the device may further comprise a handle extending along the elongate support.

Alternatively or additionally to any of the examples above, two or more of the plurality of connectors may be c-clips.

In a further example, a method of applying a flexible elongate member to a shaft of an endoscope may further comprise positioning the shaft of the endoscope through a helical configuration of the flexible elongate member, wherein the flexible elongate member in the helical configuration is coupled with a plurality of connectors along an elongate support, securing an end of the flexible elongate member relative to an end of the shaft of the endoscope, and pulling the elongate support along the shaft of the endoscope in a direction away from the end of the shaft to apply the flexible elongate member to the shaft.

Alternatively or additionally to any of the examples above, the helical configuration may be a first helical configuration and the flexible elongate member may be applied to an outer surface of the shaft in a second helical configuration as the elongate support is pulled along the shaft.

Alternatively or additionally to any of the examples above, the end of the flexible elongate member may be secured to a stop configured to couple with the end of the shaft of the endoscope and securing the end of the flexible elongate member relative to the end of the shaft of the endoscope may comprise coupling the stop with the end of the shaft of the endoscope.

Alternatively or additionally to any of the examples above, coupling the flexible elongate member to the elongate support to support the flexible elongate member in the helical configuration.

Alternatively or additionally to any of the examples above, pulling the elongate support along the shaft in the direction away from the end of the shaft may sequentially disengage the plurality of connectors from the elongate support.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of a distal end of an illustrative endoscope shaft with an elongate member extending along the shaft;

FIG. 3. depicts a schematic view of a distal end of an illustrative endoscope shaft with an elongate member helically wrapped along the shaft;

FIG. 4 depicts a schematic perspective view of an illustrative tool for applying an elongate member to an elongate shaft;

FIGS. 5A-5C depict a schematic technique for applying an elongate member to an elongate shaft using the tool depicted in FIG. 4;

FIG. 6 depicts a schematic perspective view of an illustrative tool for applying an elongate member to an elongate shaft;

FIG. 7 depicts a schematic perspective view of the illustrative to depicted in FIG. 6, with an elongate member;

FIG. 8 depicts a schematic perspective view of an illustrative tool for applying an elongate member to an elongate shaft; and

FIG. 9 depicts a schematic perspective view of the illustrative tool depicted in FIG. 8, with an elongate member.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in transluminal medical procedures. However, it should be noted that reference to this particular procedure is provided only for convenience and not intended to limit the disclosure. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with a configuration, the particular feature, structure, or characteristic may be implemented in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

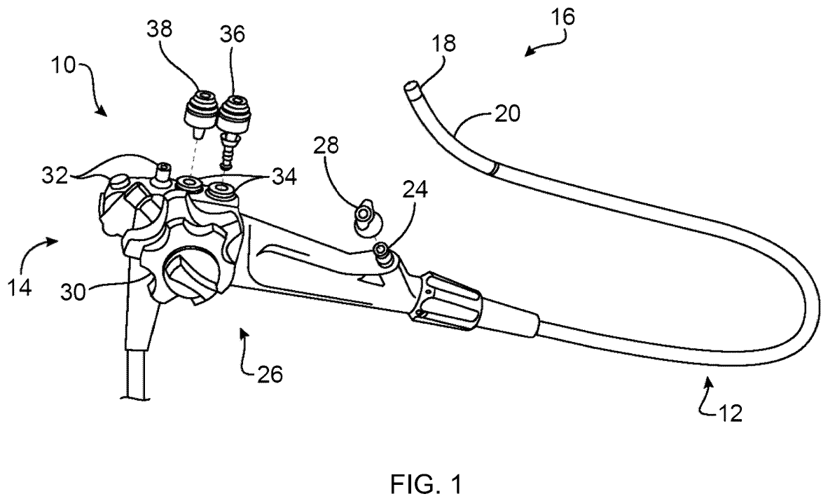

With reference to FIG. 1, an illustrative example of a medical scope, in the form of an endoscope 10, is depicted in a schematic perspective view. The endoscope 10 may include an elongate tube or shaft 12 that is configured to be inserted into a subject (e.g., a patient).

The endoscope 10 may include a proximal portion 14 and a distal portion 16. The distal portion 16 of the endoscope 10 may house or be operatively coupled with an imager (e.g., CCD or CMOS imager) (not shown). In some cases, a light source of an endoscope system including the endoscope 10 may feed illumination light to a distal portion 16 of the endoscope 10.

The endoscope shaft 12 may include a distal tip 18 (e.g., a distal tip unit adapted to be inserted into a body cavity of a patient) provided at the distal portion 16 of the shaft 12 and a flexible bending portion 20 proximal of a distal tip 18. The flexible bending portion 20 may include an articulation joint (not shown) to assist with steering the distal tip 18, but this is not required.

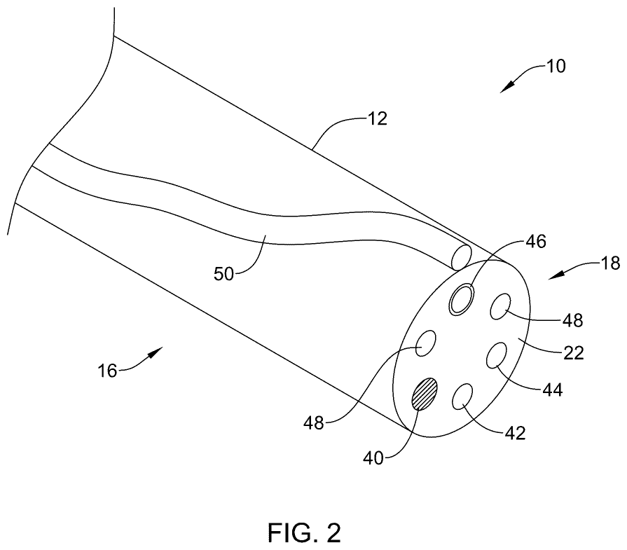

The distal tip 18 of the endoscope 10 may include an end face (e.g., an end face 22 as depicted in FIG. 2). In some cases, the end face may have a gas/lens wash nozzle for supplying gas to insufflate the interior of the patient at the treatment area and for supplying water to wash a lens covering the imager, an irrigation opening that may supply irrigation fluid to the treatment area (e.g., a target site) of the patient, one or more illumination windows that convey illumination light to the treatment area, and an opening to a working channel extending along the shaft 12 for passing tools to the treatment area. In some examples, the working channel may extend along the shaft 12 to a proximal channel opening 24 positioned distal to an operating handle 26 (e.g., a proximal handle) of the endoscope 10. A biopsy valve 28 may be utilized to seal the channel opening 24 against unwanted fluid egress.

The operating handle 26 may be provided with one or more knobs 30 for providing steering, such as remote 4-way steering of the distal tip, via wires connected to the articulation joint in the flexible bending portion 20 (e.g., one knob may control up-down steering and another knob may control left-right steering). A plurality of video switches 32 for remotely operating a video processing unit may be arranged on the handle 26 at the proximal portion 14 of the endoscope 10.

The handle 26 may be provided with dual valve locations 34. One of the valve locations 34 may have or receive a gas/water valve 36 for operating an insufflating gas and lens water feed operation. A gas supply line and a lens wash supply line may run distally from the gas/water valve 36 along the shaft 12 and converge at the distal tip 18 proximal to the gas/wash nozzle, but this is not required.

The other valve location 34 may have or receive a suction valve 38 for operating a suction operation. A suction supply line may run distally from the suction valve 38 along the shaft 12 to a junction point in fluid communication with the working channel of the endoscope 10.

Although an illustrative endoscope 10 is described with respect to FIG. 1, other suitable configurations of the endoscope 10 are contemplated and one or more other suitable delivery devices may be utilized with the concepts discussed herein. In some examples, an illustrative configuration of a delivery device may include the elongate shaft 12 and the handle 26, along with other suitable components for using the delivery device (e.g., the endoscope 10, other suitable medical scopes, and/or one or more other suitable delivery devices) within the subject.

FIG. 2 depicts a schematic view of the elongate shaft 12 at the distal portion 16 of the endoscope 10. As depicted in FIG. 2, the elongate shaft 12 may include one or more working channels 40 with an opening in the end face 22. Further, the end face 22 may include openings for one or more other channels extending through the elongate shaft 12 of the endoscope 10 including, but not limited to, one or more suction/aspiration channels 42, one or more irrigation channels 44, one or more imaging channels 46, one or more light guide channels, and/or one or more other suitable channels.

In some cases, one or more elongate members 50 (e.g., one or more flexible elongate members and/or other suitable elongate members) may extend along the elongate shaft 12 between the distal portion 16 of the endoscope 10 and the proximal portion 14 of the endoscope 10. Although not required, a distal end of the elongate member 50 may be coupled to a stop (e.g., a cap) or other suitable component at a distal portion 16 of the endoscope 10 and coupled with or proximate the handle 26 at the proximal portion 14 of the endoscope 10. The elongate member(s) 50 may by any suitable type of elongate member 50 including, but not limited to coils, cabling, tubing, auxiliary working channels, aspiration channels, gas/liquid channels, light channels, optical channels, and/or other suitable types of elongate members 50. Although the elongate members 50 are depicted in the Figures and discussed herein as extending along the endoscope 10, it is contemplated that the devices herein (e.g., the elongate member 50, etc.) may be used with medical scopes other than the endoscope 10, or with elongate members of other medical devices (including, but not limited to, elongate members insertable into a patient's body, such as, without limitation, transluminally), the present disclosure not being limited in this regard.

The elongate members 50 may extend along the elongate shaft 12 of the endoscope 10 during a procedure without a securing mechanism along the elongate shaft 12 or, alternatively, with a sheath (e.g., a silicone sheath and/or other suitable type of sheath) or other suitable securing mechanism applied over the elongate member 50 and the elongate shaft 12 to restrict or constrain relative movement of the elongate members 50 relative to the elongate shaft 12. When the elongate member(s) 50 are not constrained relative to the elongate shaft 12 (e.g., as depicted in FIG. 2), tissue may be pinched by the elongate member(s) 50 and/or between the elongate member(s) 50 and the elongate shaft 12 during deployment, extraction, and/or adjustment of the endoscope 10 within the subject (e.g., the patient) (e.g., as a result of relative movement between the elongate member 50 and the elongate shaft 12).

Utilizing a sheath to constrain the elongate member 50 relative to the elongate shaft 12 may add to an overall diameter of the elongate shaft 12 and the elongate member 50, which may affect a compatibility of the elongate shaft 12 with over-tubes and thus, increase a likelihood of tissue trauma and/or scraping during navigation of the elongate shaft 12 to a target site and/or extraction of the elongate shaft 12 from the body of the subject. Further, utilizing a sheath extending along the elongate shaft 12 may hinder a mobility of the elongate shaft 12 (e.g., may stiffen and/or reduce flexibility of the elongate shaft 12) and/or the elongate member 50, which may reduce an ability of the elongate shaft 12 to be used for navigating tortuous paths.

Wrapping the elongate member 50 extending along the elongate shaft 12 helically around the elongate shaft 12 may facilitate securing the elongate member 50 relative to the elongate shaft 12. The helical wrapping may be facilitated by a tool that facilitates securing the elongate member 50 relative to the elongate shaft 12. Such securing of the elongate member 50 relative to the elongate shaft 12 may only minimally increases an overall diameter of the endoscope 10 along the elongate shaft 12, while maintaining mobility of the endoscope 10 (e.g., flexibility of the elongate shaft 12 and the elongate member 50). The tool may facilitate achieving quick and straightforward setup of the elongate member 50 relative to the elongate shaft 12.

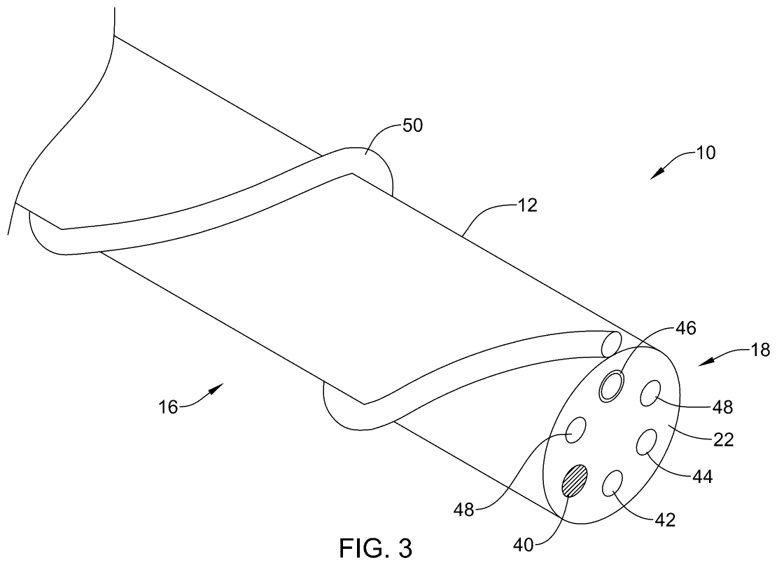

FIG. 3 depicts a schematic perspective view of the elongate member 50 helically wrapped around the elongate shaft 12. However, helically wrapping the elongate member 50 around the elongate shaft 12 may be cumbersome due to a variety of factors including, but not limited to, the lengths of the elongate shaft 12 and the elongate member 50, the rotational stiffness of the elongate shaft 12 and/or the elongate member 50, and/or other suitable factors. As such, a tool may be utilized to facilitate wrapping the elongate member 50 helically around the elongate shaft 12.

The tool may be configured to receive and maintain the elongate member 50 in an initial or first helical configuration until the elongate member 50 is applied to an outer surface of the elongate shaft 12 such that the elongate member 50 has a subsequent or second helical configuration. The tool may enable users to constrain cabling quickly and efficiently along the elongate shaft 12 of the endoscope 10.

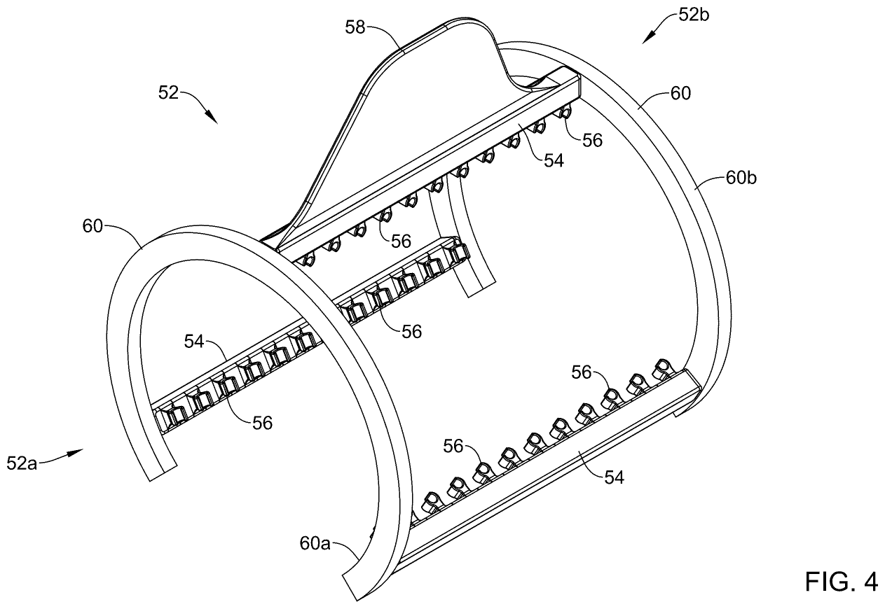

FIG. 4 depicts a schematic perspective view of an illustrative configuration of a tool 52 configured to facilitate applying the elongate member 50 to the elongate shaft 12. The tool 52 may be configured to receive and maintain the elongate member 50 in an initial helical configuration until the elongate member is applied to an outer surface of the elongate shaft 12 such that the elongate member 50 has a subsequent helical configuration. The initial first configuration of the elongate member 50 in the tool 52 may have an inner diameter configured to receive or fit over an outer surface of the elongate shaft 12.

The tool 52 may include one or more elongate supports 54 (e.g., spines, etc.), a plurality of connectors 56 configured to connect or couple with the elongate member 50, and a handle 58. The tool 52, however, may have one or more other suitable components.

The tool 52 may include any suitable number of elongate supports 54. For example, the tool 52 may include a single elongate support 54, two elongate supports 54, three elongate supports 54, five elongate supports 54, and/or any other suitable number of supports. In one example, the tool 52 may include three elongate supports 54, as depicted in FIG. 4, but this is not required.

When the tool 52 includes two or more elongate supports 54, the elongate supports 54 may be spaced apart from one another in any suitable manner configured to support the elongate member 50 within the connectors 56. In some cases, the two or more elongate supports 54 may be equally circumferentially spaced about a circumference of an imaginary cylinder so as to be configured to be equally spaced around a helix formed by the elongate member 50 coupled with the connectors 56.

When the tool 52 includes two or more elongate supports 54, the tool 52 may also include one or more connecting segments 60 configured to couple the two or more elongate supports 54. Any suitable number of connecting segments 60 may be utilized. In some examples, the tool 52 may include two connecting segments 60 when the tool 52 includes a plurality of elongate supports 54. In one example and as depicted in FIG. 4, the tool 52 may include three circumferentially spaced apart elongate supports 54 and two connecting segments 60, with a first connecting segment 60a at a first end 52a of the tool 52 (e.g., a distal end of the tool 52) and a second connecting segment 60b at a second end 52b of the tool 52 (e.g., a proximal end of the tool 52), but this configuration is not required and the connecting segments 60 may be configured in other suitable manners.

The connecting segment(s) 60, when included, may have any suitable configuration. For example, a shape of the connecting segments(s) 60 may have linear portions, arced portions, a circular or annular shape, and/or other suitable configurations. In some examples, the connecting segments 60 may have an arced configuration, as depicted for example in FIG. 4.

The connectors 56 may have any suitable configuration configured to connect to the elongate member 50 (e.g., releasably couple with the elongate member 50 and/or connect in one or more other suitable manners) and maintain the elongate member 50 in a helical configuration until the elongate member 50 is to be released from the connectors 56. In some cases, the connectors 56 may be configured to connect with the elongate member 50 at two or more locations along the elongate member 50 when the elongate member 50 is in a helical configuration (e.g., the elongate member 50 is entirely or at least partially in the first helical configuration).

Example suitable types of connectors 56 include, but are not limited to, snap connectors, clip connectors, clasp connectors, C-shaped connectors, releasable adhesive connectors, belt or band connectors, and/or other suitable types of connectors. In some examples, the different connectors 56 utilized in the tool 52 may have the same or different connection types. In some examples and as depicted in FIG. 4, the connectors 56 may all be C-shaped clip connectors 56 (e.g., C-clips) configured to receive the elongate member 50 with a snap fit. Although the connectors 56 may face any suitable direction, in some examples the C-shaped clip connectors 56 may be configured to face the first end 52a of the tool 52 such that less force may be required to disengage the elongate member 50 from the connectors 56 as the tool 52 is adjusted along the elongate shaft 12 from a distal to proximal direction than if the connectors 56 faced the second end 52b of the tool, but this is not required.

The connectors 56 may be supported by the elongate supports 54 in any suitable manner. In some examples, the connectors 56 may be longitudinally spaced apart from one another in a row along the elongate support(s) 54 (e.g., the connectors 56 along the elongate support 54 may be configured to couple with the elongate member 50 at a same circumferential location, longitudinally spaced when the elongate member 50 is in a helical configuration), but this is not required and the connectors 56 supported by the elongate support 54 may be laterally (e.g., circumferentially) offset from one another, longitudinally formed with one another, and/or configured with respect to the elongate support 54 in one or more other suitable manners. When the connectors 56 are longitudinally spaced apart from one another, the spacing may be based on a desired pitch of the turns or loops of first helical configuration, but this is not required. When the tool 52 includes three elongate supports 54 (e.g., as depicted in FIG. 4), the elongate supports may be equally circumferentially spaced from one another (e.g., spaced approximately one hundred twenty degrees apart from one another) and two or more connectors 56 may extend in a first row along a first elongate support 54, two or more connectors 56 may extend in a second row along a second elongate support 54, and two or more connectors 56 may extend in a third row along a third elongate support 54. Other suitable configurations are contemplated.

Any suitable number of connectors 56 having any suitable spacing may be utilized to support each loop of the elongate member 50 in the first helical configuration as the elongate member 50 is applied to the elongate shaft 12. In some cases, the number of connectors 56 and spacing of the connectors 56 may depend on one or more factors including, but not limited to, a number of elongate supports 54 that are utilized, a length of each elongate support 54, a desired number of loops in the first helical configuration of the elongate member 50 when positioned within the tool 52, a desired spacing between loops of the first helical configuration, a pitch of the loops of the first helical configuration, a diameter of the loops, a length of the elongate member 50, and/or other suitable factors. In some cases, the number of connectors 56 and/or the spacing of the connectors 56 may set the diameter and pitch of the loops of the first helical configuration of the elongate member 50, which may be determined to achieve a desired pitch of the elongate member 50 in the second helical configuration around the elongate shaft 12 of a known diameter. In one example configuration and as depicted in FIG. 4, each elongate support 54 of the three elongate supports 54 may include or otherwise support twelve connectors 56 for a total of 36 connectors, but other suitable number of connectors 56 may be utilized as desired.

The handle 58 may have any suitable configuration. In some cases, the handle 58 may be configured to be grasped by a user to adjust the tool 52 in distal to proximal direction. Example suitable configurations for the handle 58 may include, but are not limited to, a fin configuration, a looped configuration configured to receive fingers of a user, indents for receiving fingers of a user, an orientation indicating a direction of use of the tool 52, and/or other suitable configurations. In some examples and as depicted in FIG. 4, the handle 58 may be a fin-type handle extending radially outward from a middle or center elongate support 54 of three equally spaced elongate supports 54 and may have a directional lean toward the second end 52b of the tool 52 and/or other suitable direction lean (e.g., the handle 58 may be angled toward the second end 52b or be angled in one or more other suitable manners). Further, although only a single handle 58 extending from a single elongate support 54 is depicted in FIG. 4, the tool 52 may include multiple handles 58 and/or the handle 58 may extend from multiple elongate supports 54.

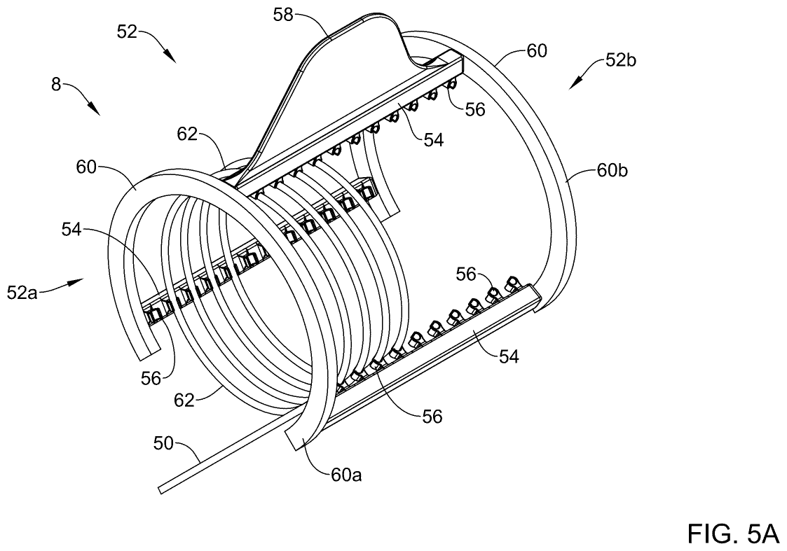

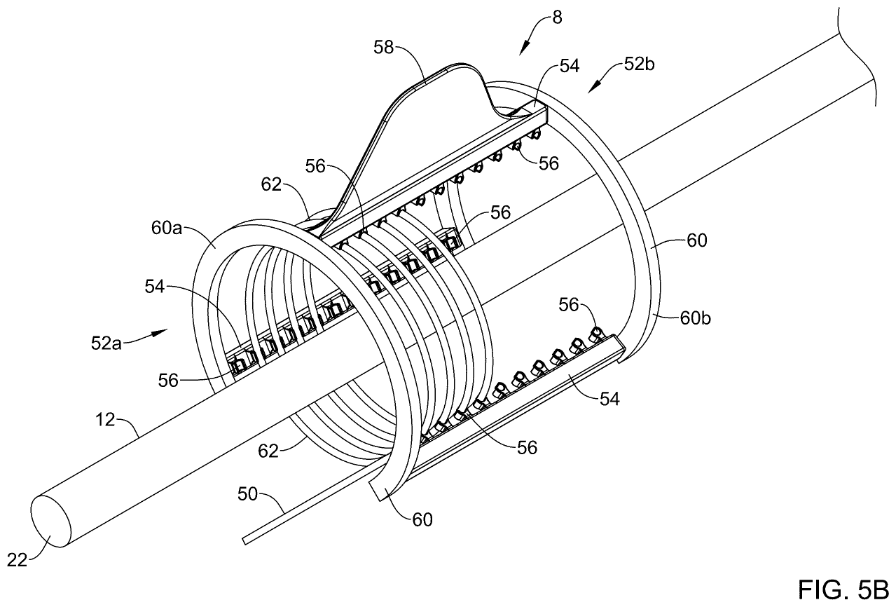

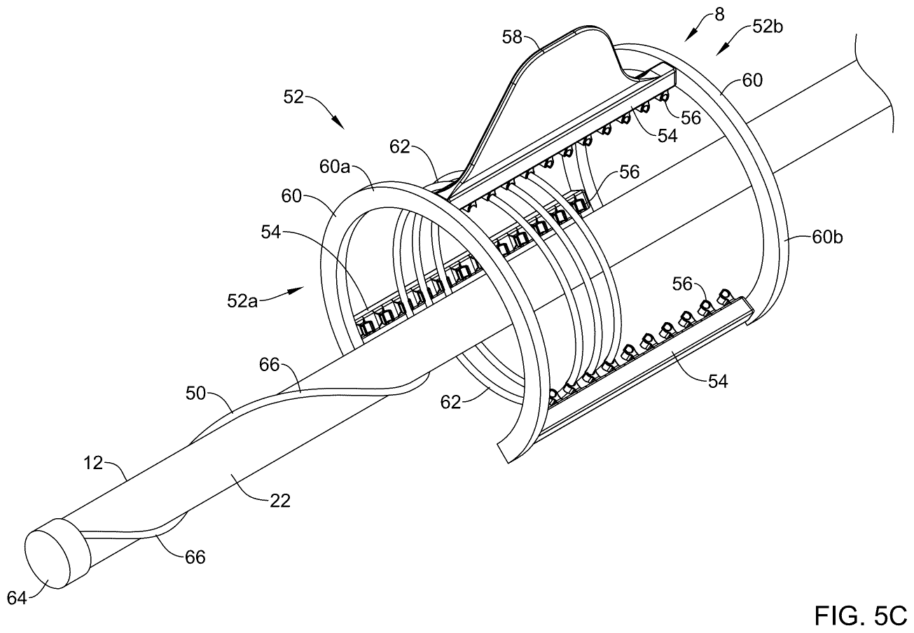

FIGS. 5A-5C depict a system 8 for applying the elongate member 50 to the elongate shaft 12 of the endoscope 10 and a method or technique for applying the elongate member 50 to the elongate shaft 12 of the endoscope 10. In some examples, the system 8 may include the elongate member 50, the tool 52, and/or one or more additional or alternative components.

As depicted in FIG. 5A, the system may include the tool 52 and the elongate member 50 received in and coupled with the connectors 56. Although other suitable configurations of the tool are contemplated, the tool 52 may include three elongate supports 54 with connectors 56 longitudinally spaced apart from one another on the elongate supports 54 forming three rows of connectors circumferentially spaced apart from one another. The elongate member 50 may be received in the connectors 56 via snap-fit or clip-fit and/or via one or more other suitable connecting techniques.

As discussed, the elongate member 50 may have any suitable length and when the elongate member 50 is in the first helical configuration and connected with the connectors 56 of the tool 52, the elongate member 50 may or may not have a sufficient length to connect to all of the connectors 56 in the tool 52. As depicted in FIG. 5A for example, not all of the connectors 56 are coupled or connected with the elongate member 50.

In some cases, the elongate member 50 may be pre-coupled with the connectors 56, such that the system 8 is provided to a user (e.g., in a package) with the elongate member 50 in the first helical configuration and coupled with the connectors 56 of the tool 52. In other instances, the tool 52 may be provided to a user without the elongate member 50 positioned in the tool 52 in the first helical configuration. When the tool 52 is provided without the elongate member 50 coupled to the connectors 56, a user may couple the elongate member 50 with the connectors 56 by snapping the elongate member 50 into the connectors 56, for example, to position and maintain the elongate member 50 in the first helical configuration.

FIG. 5A depicts the elongate member 50 coupled with consecutive connectors 56. However, to create a desired pitch, to reduce a force needed to disconnect the elongate member 50 from the connectors 56 of the tool 52, and/or for one or more additional or alternative purposes, one or more connectors 56 may be skipped as the elongate member 50 is coupled with the connectors 56.

FIG. 5B depicts the system 8 with the elongate shaft 12 of the endoscope 10 positioned within the elongate member 50 in the first helical configuration on the tool 52. In some examples, the elongate shaft 12 may be inserted into the second end 52b of the tool 52 connected with the elongate member 50 and passed through the tool 52 such that the distal end of the elongate shaft 12 extends through the first end 52a of the tool 52.

A distal end of the elongate member 50 may be free from or otherwise not engaged with the connectors 56 of the tool 52. In some examples, the distal end of the elongate member 50 may be free from the tool 52 so as to be configured for coupling with and/or fixing with a distal end of the elongate shaft 12.

FIG. 5C depicts the system 8 with a portion of the elongate member 50 released from the connectors 56 of the tool 52 and wrapped around the elongate shaft 12 in the second helical figuration having loops 66 (e.g., coils), where the loops 66 of the second helical configuration have a different pitch than the first helical configuration. In some examples, the pitch of the second helical configuration may be greater than the pitch of the first helical configuration, but this is not required. In some examples, the pitch of the elongate member 50 may be determined based on a diameter and pitch of the loops 62 in the first helical configuration.

To wrap the elongate member 50 around the elongate shaft 12, a user may fix or secure a distal end or other suitable portion of the elongate member 50 at a location of the elongate shaft 12 distal of the tool 52 (e.g., at a distal end of the elongate shaft 12). Once the elongate member 50 is fixed relative to the elongate shaft 12, a user may pull (e.g., withdraw) the tool 52 and thus the elongate support(s) 54 (e.g., a user may pull the tool 52 while grasping the handle 58 and/or grasping the tool 52 in one or more additional or alternative manners) in a proximal direction (e.g., in a direction from the first end 52a of the tool 52 to the second end 52b of the tool 52 and away from the distal end of the elongate shaft 12), which may cause the elongate member 50 to release from the connectors 56 (e.g., in a sequential manner) and wrap around the elongate shaft 12 and form the loops 66 having a desired pitch. Fixing the distal end or other suitable portion of the elongate member 50 relative to the elongate shaft 12 may facilitate maintaining a position of the distal end or other suitable portion of the elongate member 50 relative to the elongate shaft 12 as the tool 52 and the elongate support(s) 54 are moved longitudinally along the elongate shaft 12 away from the distal end of the elongate shaft 12.

The elongate member 50 (e.g., the distal end of the elongate member 50) may be fixed (e.g., releasably fixed, permanently fixed, etc.) with respect to the elongate shaft 12 (e.g., with respect to the distal end of the elongate shaft 12) using a stop 64. Example suitable configurations of the stop 64 for fixing the elongate member 50 with respect to the elongate shaft 12 include, but are not limited to, one or more caps coupled with or configured to be coupled with a distal end of the elongate shaft 12 (e.g., as depicted in FIG. 5C), one or more adhesives, one or more wraps or bands, one or more silicone wraps, one or more clips, one or more pieces of tape, and/or one or more other suitable configurations.

Once the elongate member 50 has been applied to the elongate shaft in a desired configuration, the user may apply one or more fixation components to the elongate member 50 and the elongate shaft 12 (e.g., apply the fixation components intermittently) such that the elongate member 50 maintains a desired configuration (e.g., the second helical configuration) about the elongate shaft 12. In some cases, the fixation components may be similar to the stop 64, but this is not required. Example suitable manners for fixing the elongate member 50 with respect to the elongate shaft 12 along a length of the elongate shaft 12 may include, but are not limited to, one or more caps, one or more adhesives, one or more wraps or bands, one or more silicone wraps, one or more clips, one or more pieces of tape, and/or may be secured relative to the elongate shaft 12 in one or more other suitable manners.

Although fixation components may facilitate maintaining a desired configuration of the elongate member 50 relative to the elongate shaft 12, the fixation components may not be required. For example, in some cases, the forces applied to the elongate shaft 12 by the elongate member 50 in the second helical configuration may be sufficient to cause the elongate member 50 to maintain its configuration about the elongate shaft 12.

With the elongate member 50 applied to the elongate shaft 12 in the second helical configuration and the fixation components, optionally, applied to the elongate member 50 and the elongate shaft 12, the elongate shaft 12 may maintain a desired amount of flexibility and a smaller outer diameter may be maintained relative to when a sleeve applied along the elongate shaft 12 is utilized to maintain the elongate member 50 relative to the elongate shaft 12. Further, the tight helical winding of the elongate member 50 along the elongate shaft 12 which may be achieved in accordance with the present disclosure may prevent or mitigate tissue pinching between the elongate member 50 and the elongate shaft 12, which may mitigate potential tissue trauma during positioning of the elongate shaft 12 within the subject and/or extraction of the elongate shaft 12.

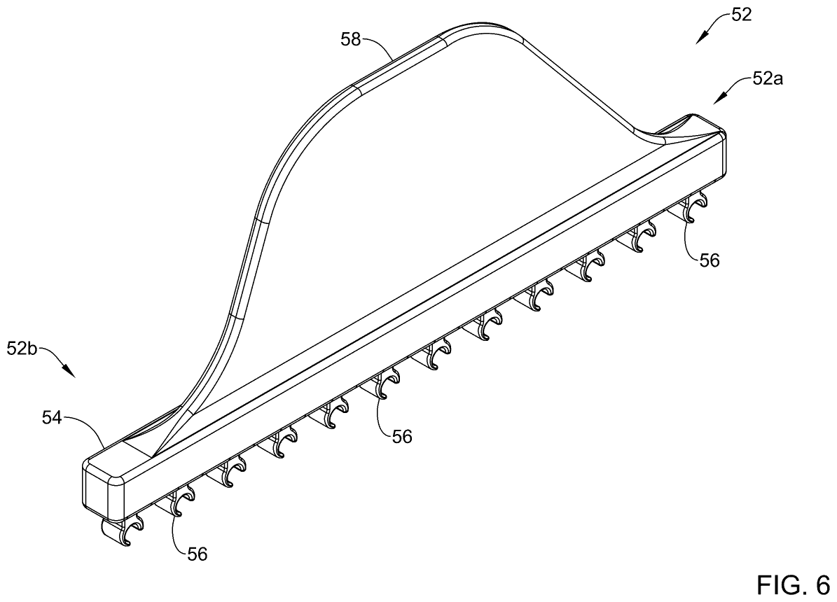

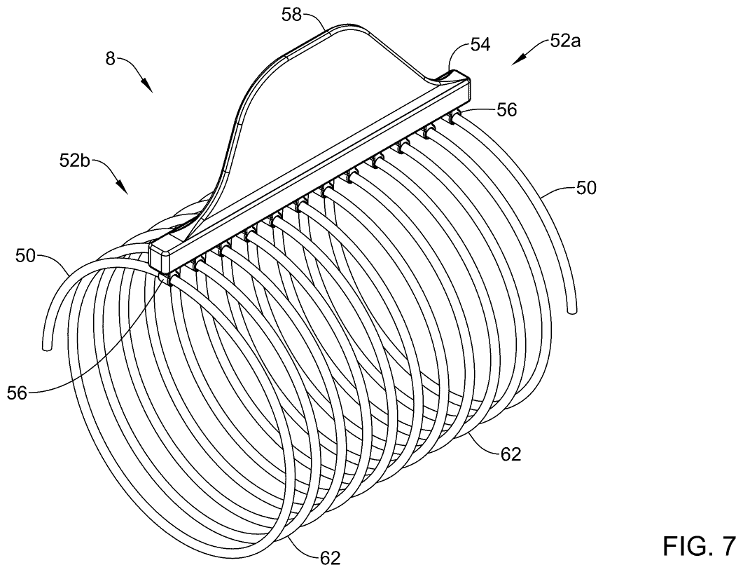

FIGS. 6 and 7 depict a schematic perspective view of an illustrative configuration of a tool 52 configured to facilitate applying the elongate member 50 to the elongate shaft 12, where the tool 52 has a single elongate support 54 with the connectors 56 arranged (e.g., longitudinally spaced apart) in a single longitudinally extending row. The tool 52 of the system 8 may be configured to receive and maintain the elongate member 50 in an initial or first helical configuration with loops 62 having a desired pitch, as depicted for example in FIG. 7, until the elongate member 50 is applied to an outer surface of the elongate shaft 12 such that the elongate member 50 has a subsequent or second helical configuration. The first helical configuration of the elongate member 50 in the tool 52 may have an inner diameter configured to receive or fit over an outer surface of the elongate shaft 12.

As depicted in FIGS. 6 and 7, the elongate support 54, the connectors 56, and the handle 58 may be similar to the similar components of the configuration of the tool 52 depicted in FIG. 4 with three elongate supports 54. However, this is not required and one or more of the elongate supports 54, the connectors 56, and the handle 58 of the configuration of the tool 52 with a single elongate support 54, or other suitable number of elongate supports 54, may be configured in a manner that differs from the configuration with three elongate supports 54, as depicted in FIG. 4.

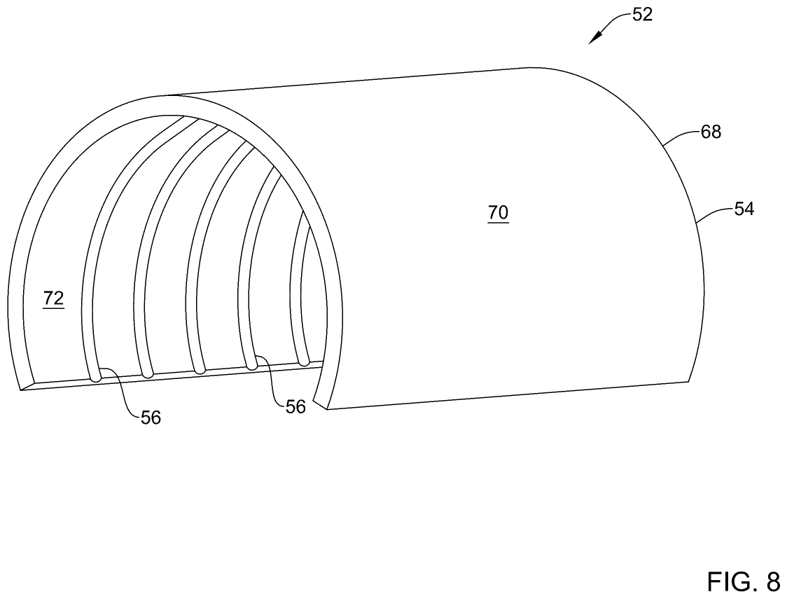

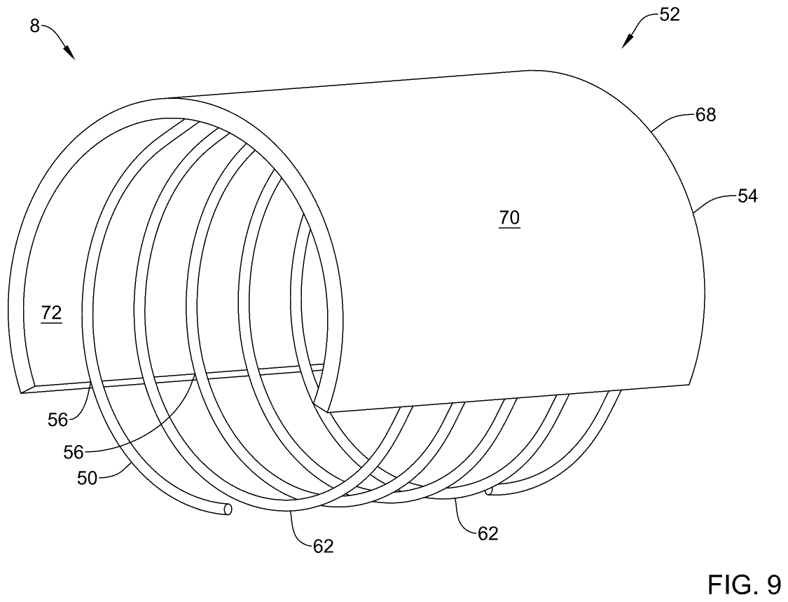

FIGS. 8 and 9 depict a schematic perspective view of an illustrative configuration of a tool 52 configured to facilitate applying the elongate member 50 to the elongate shaft 12, where the elongate support 54 of the tool 52 may be formed from an elongate sheet 68 of material. The tool 52 of the system 8 may be configured to receive and maintain the elongate member 50 in an initial or first helical configuration with loops 62 having a desired pitch, as depicted for example in FIG. 9, until the elongate member 50 is applied to an outer surface of the elongate shaft 12 such that the elongate member 50 has a subsequent or second helical configuration. The first helical configuration of the elongate member 50 in the tool 52 may have an inner diameter configured to receive or fit over an outer surface of the elongate shaft 12.

The sheet 68 of material may be formed from any suitable materials. Example suitable materials from which the sheet 68 may be formed include, but are not limited to, polymers, flexible polymers, metals, plastic, rubber, silicone, fabric, and/or other suitable materials.

The elongate support 54 formed from the sheet 68 may have any suitable configuration. In some examples, the sheet 68 may be formed in an arced configuration (e.g., C-shaped), as depicted in FIGS. 8 and 9, but other suitable configurations are contemplated.

An outer surface 70 of the sheet 68 may be configured to be grasped by a user. The outer surface 70 may be devoid of any grips or markings, as depicted in FIGS. 8 and 9, and allow a user to grasp the tool 52 by applying a radially inward pressure on opposing sides of the sheet 68. However, the outer surface 70 may include one or more features configured to facilitate grasping the tool 52 and adjusting the tool 52 relative to the elongate shaft 12 that may include, but are not limited to, the handle 58, one or more indents, one or more bumps or protrusions, one or more openings, a slip-resistant coating, and/or one or more other suitable features for facilitate grasping the tool 52.

An inner surface 72 of the sheet 68 (e.g., a surface opposing the outer surface 70) may include and/or define the connectors 56. In some examples, the connectors 56 may be formed in the inner surface 72, as depicted in FIGS. 8 and 9, but this is not required and the connectors 56 may extend from the inner surface in a manner similar to how the connectors 56 extend from the elongate supports 54 discussed above and depicted in FIGS. 4-7, and/or one or more other suitable manners.

The connectors 56 may be configured to extend an entire width or circumference of the sheet 68, but this is not required. Additionally or alternatively, one or more of the connectors 56 on the inner surface 72 of the sheet 68 may extend less than an entire width or circumference of the sheet 68. In some examples, the connectors 56 on the inner surface 72 of the sheet 68 may be circumferentially spaced from one another similar to as discussed above when the tool 52 may include a plurality of elongate supports 54.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for applying a flexible elongate member to a shaft of an endoscope, the system comprising:
an elongate support;
a plurality of connectors longitudinally spaced apart along the elongate support; and
a flexible elongate member, and
wherein the plurality of connectors are releasably coupled with the flexible elongate member at two or more locations along the flexible elongate member when the flexible elongate member is in a helical configuration.

2. The system of claim 1, wherein the plurality of connectors are releasably coupled with the flexible elongate member at a same circumferential location, longitudinally spaced apart along the flexible elongate member in the helical configuration.

3. The system of claim 1, further comprising:
a stop secured to an end of the flexible elongate member, and
wherein the stop is configured to couple with an end of the shaft of the endoscope about which the flexible elongate member is applied and maintain a position of the end of the flexible elongate member relative to the shaft as the elongate support is moved longitudinally along the shaft and away from the end of the shaft.

4. The system of claim 3, wherein the stop is one of an endoscope clip and a cap.

5. The system of claim 1, further comprising:
a plurality of longitudinally extending rows of the plurality of connectors, the plurality of longitudinally extending rows are circumferentially spaced apart from one another.

6. The system of claim 5, wherein the plurality of longitudinally extending rows of connectors comprise three longitudinally extending rows of connectors with each row circumferentially spaced equally apart about the flexible elongate member in the helical configuration.

7. The system of claim 1, wherein the plurality of connectors are arranged in a single longitudinally extending row along the elongate support.

8. The system of claim 1, wherein the elongate support is an elongate sheet of material defining the plurality of connectors.

9. The system of claim 1, further comprising:
a handle extending along the elongate support.

10. The system of claim 1, wherein one or more connectors of the plurality of connectors are c-clips.

11. A device, comprising:
an elongate support; and
a plurality of connectors, wherein two or more of the plurality of connectors are longitudinally spaced apart in a row along the elongate support, and
wherein the plurality of connectors are configured to be releasably coupled with a flexible elongate member at two or more locations along the flexible elongate member when the flexible elongate member is in a helical configuration.

12. The device of claim 11, wherein the elongate support is a first elongate support and the row along the elongate support is a first row of the plurality of connectors extending along the elongate support and the device further comprises:
two or more of the plurality of connectors in a second row of the plurality of connectors extending along a second elongate support; and
two or more of the plurality of connectors in a third row of the plurality of connectors extending along a third elongate support, and
wherein the first elongate support, the second elongate support, and the third elongate support are circumferentially spaced one hundred twenty degrees apart from one another.

13. The device of claim 11, wherein the elongate support is an elongate sheet of material defining the plurality of connectors.

14. The device of claim 11, further comprising:
a handle extending along the elongate support.

15. The device of claim 11, wherein two or more of the plurality of connectors are c-clips.

16. A method of applying a flexible elongate member to a shaft of an endoscope, the method comprising:
positioning the shaft of the endoscope through a helical configuration of the flexible elongate member, wherein the flexible elongate member in the helical configuration is coupled with a plurality of connectors along an elongate support;
securing an end of the flexible elongate member relative to an end of the shaft of the endoscope; and
pulling the elongate support along the shaft of the endoscope in a direction away from the end of the shaft to apply the flexible elongate member to the shaft.

17. The method of claim 16, wherein:
the helical configuration is a first helical configuration; and
the flexible elongate member is applied to an outer surface of the shaft in a second helical configuration as the elongate support is pulled along the shaft.

18. The method of claim 16, wherein the end of the flexible elongate member is secured to a stop configured to couple with the end of the shaft of the endoscope and securing the end of the flexible elongate member relative to the end of the shaft of the endoscope comprises:
coupling the stop with the end of the shaft of the endoscope.

19. The method of claim 16, further comprising:
coupling the flexible elongate member to the elongate support to support the flexible elongate member in the helical configuration.

20. The method of claim 16, wherein pulling the elongate support along the shaft in the direction away from the end of the shaft sequentially disengages the plurality of connectors from the elongate support.
